(19) United States
(12) Patent Application Publication
(10) Pub. No.: US 2018/0000447 A1
(43) Pub. Date: Jan. 4, 2018

(54) PORTABLE ULTRASONIC MEASURING DEVICE SUITABLE FOR MEASURING PELVIC TILT

(71) Applicants: UNIVERSITE DE BRETAGNE OCCIDENTALE, Brest (FR); CENTRE HOSPITALIER REGIONAL ET UNIVERSITAIRE DE BREST, Brest Cedex (FR)

(72) Inventors: Eric Stindel, Locmaria Plouzane (FR); Manuel Urvoy, Brest (FR); Guillaume Dardenne, Montreuil Le Gast (FR); Julien Leboucher, Brest (FR); Shaban Almouahed, Brest (FR)

(21) Appl. No.: 15/544,807

(22) PCT Filed: Dec. 17, 2015
(86) PCT No.: PCT/EP2015/080361

§ 371 Date: Jul. 19, 2017

(30) Foreign Application Priority Data
Jan. 19, 2015 (FR) 1550416

(51) Int. Cl. A61B 8/00; A61B 8/08; A61F 2/46

(52) U.S. Cl. CPC A61B 8/4245; A61B 8/4218; A61F 2/4657; A61B 8/5223; A61B 8/0875; A61B 8/4477; A61B 8/4427; A61F 2002/4668; A61B 8/462

(57) ABSTRACT

An ultrasound measuring device includes: a support bearing two ultrasound probes movable relative to each other by slide link, each of the two probes being movable relative to the support by ball-joint link, wherein the probes are capable of simultaneously acquiring two ultrasound images. The device includes a first set of measuring elements to measure a relative positioning of the probes, including one travel sensor and at least two orientation sensors. The device includes a second set of measuring elements to measure a positioning of the device relative to a reference plane, including at least one orientation sensor. The device localizes at least one point of interest on each of the two ultrasound images, and processes data coming from the first and second measuring elements, delivering a relative spatial position of the points of interest located in the images.

### 1. FIELD OF THE INVENTION

The field of the invention is that of ultrasound measuring devices. More particularly, the invention relates to an ultrasound measuring device that is particularly suited to measuring an individual's pelvic tilt but it can of course also find other medical applications. For the sake of simplification, however, we shall strive first of all, here below, to describe the invention in the special context of total hip arthoplasty or replacement.

### 2. PRIOR ART AND ITS DRAWBACKS

Surgical operations for total hip replacement concern more than 120 000 individuals per year in France. Owing to the ageing of the population, the incidence of these operations is likely to increase constantly in years to come.

A total hip prosthesis generally comprises two parts: a first part attached to the femur, called a femoral part and comprising a stem introduced into the femur fitted with an essentially spherical head and an acetabulum designed to receive the femoral head. The acetabulum, also called a cup or shell when it is semispherical, takes position in the corresponding housing (the anatomical acetabulum) of the iliac or pelvic bone.

The implanting of a prosthetic device by a surgeon is a relatively complex operation since the femoral part, and to an even greater extent the acetabulum, must be placed in an optimized manner, especially to prevent the prosthesis from getting dislocated during high-amplitude motions.

According to classic methods, the pelvis is palpated to locate the three points of the anterior pelvic plane (APP). This anterior pelvic plane (also called the Lewinneck plane) is a reference plane classically used in hip surgery. It is defined by the two iliac spines and the pubic symphysis. This plane enables the prosthetic acetabulum to be oriented suitably, in terms of inclination and anteversion.

The surgeon then inserts the acetabulum, or cup, at the tip of a tool called an impactor. He handles this cup in such a way as to place it so that it has an inclination of 45° and an anteversion of 15° relative to the anterior pelvic plane.

These two values of angles are, however, mean values used by default and do not correspond to all the particular situations that are likely to be encountered.

One improvement to this approach has been proposed in the U.S. Pat. No. 6,205,411. This patent document proposes a pre-operative computerized simulation of the prosthesis using a tomography scan of the bone casing of the pelvis and the femur, done pre-operatively.

The surgeon is guided, during the operation, and by means of an internal body placed on the pelvis and the femur to carry out an operation of locating in space in order to position the acetabulum according to the result of the simulation.

This approach is efficient but has the drawback of high complexity (in terms of tomography, computer simulations etc.) which make its use limited, especially for reasons of cost.

Another approach is proposed in the patent document FR-2 865 928. This technique uses a “mega-head” placed in the acetabular cavity hollowed out in the pelvis. A processing device enables a simultaneous display of a cone of mobility and of extreme positions, as a function of the center of the cup and the geometry of the femoral prosthesis.

The surgeon can then handle the cup by means of an impactor to bring the extreme positions within the cone of mobility.

This technique is simpler than the one described in the document U.S. Pat. No. 6,205,411, and does not require preliminary measurements. However, it proves to be inadequate in practice because the measurements are made per-operatively, the individual being kept unconscious in a particular position (the supine position).

It has indeed been observed that about 13% of arthoplasty operations need revision surgery because of joint dislocation or premature wear and tear of the prosthetic elements, themselves due to a non-optimal positioning of the implants.

In “Toward a Dynamic Approach of THA Planning Based on Ultrasound”, Clinical Orthopaedics and Related Research, 467(4), 901-908, 2009, Dardenne et al. propose to take account of the pelvic dynamics proper to each individual during preoperative treatment of patients to reduce the risks of inappropriate positioning of the prosthesis. To this end, Dardenne et al. recommend the use of ultrasound measurements to determine the pelvic tilt of patients in three positions: standing, seated and supine.

The measuring apparatus used comprises especially a 3D infrared localizer and a 2D ultrasound probe equipped with retroreflective trackers, so as to be capable of being localized in a 3D volume by the infrared localizer. The 2D ultrasound probe must furthermore be calibrated according to a method of calibration based on a special phantom and the introduction of virtual motions applied to the probe, as described in the patent document FR 2 924 810.

The regions of interest are then scanned by means of the ultrasound probe and, with a dedicated interface, the user of the measuring apparatus localizes the anatomical landmarks (the anterior-superior iliac spines and the pubic symphysis) in the corresponding ultrasound images.

Although this approach is interesting in its principle, it has several drawbacks that make it complicated to use.

First of all, the ultrasound measuring apparatus presented by Dardenne et al. is bulky because it comprises, on the one hand, an ultrasound acquisition station and, on the other hand, a station for localizing the probe: indeed, before each measurement, this device requires the calibration of the ultrasound probe by means of a phantom plane to then enable the 3D localizing of the 2D points of interest situated in the ultrasound images. Such an apparatus is therefore not portable. This makes the apparatus difficult to use in day-to-day medical consultation.

In addition, the anatomical landmarks constituted by the anterior-superior spines and the pubic symphysis must be localized manually by the surgeon in the ultrasound images. This proves to be a lengthy process and is also more complicated in the seated and standing positions.

There is therefore a need for an ultrasound measuring technique adapted especially but not exclusively to the measurement of an individual's pelvic tilt, that does not have these different drawbacks of the prior art.

### 3. SUMMARY OF THE INVENTION

The invention meets this need by proposing an ultrasound measuring device that comprises:
a support bearing two ultrasound probes movable relative to each other by slide link, each of the two probes being movable relative to the support by ball-joint link, said probes being capable of simultaneously acquiring two ultrasound images;
first measuring means for measuring a relative positioning of said probes comprising one travel sensor and at least two orientation sensors;
second measuring means for measuring a positioning of said device relative to a reference plane, comprising at least one orientation sensor;
means for localizing at least one point of interest on each of said two ultrasound images;
means for processing data coming from said first and second measuring means, capable of delivering a relative spatial position of said points of interest located in said images.

Thus the invention relies on a wholly novel and inventive approach to ultrasound measurement, especially but not exclusively in the context of total hip replacement.

Indeed, the invention proposes an ultrasound measurement device enabling the simultaneous acquisition of two images corresponding to two anatomical zones of interest in the patient. This device is particularly simple to use, because the probes have six degrees of freedom relative to each other, thus enabling the device to adapt well to each patient's morphology, and providing efficient image-capturing conditions. This architecture especially enables the practitioner to easily adjust the probes on the anatomical points of interest for the measurement of the pelvic tilt.

In addition, the designing of such a measuring device removes the need to localize the probes, thus enabling the device to be portable and be used in medical consultation. Indeed, the presence of orientation and travel sensors in the measuring device makes it possible to know the position of the two probes, relative to each other and in space. When the anatomical points of interest have been localized in the images acquired by the ultrasound probes, the measuring device of the invention can then directly deduce the relative spatial positions of these anatomical points since the relative positions of the probes are known.

Finally, the measuring device of the invention proposes non-irradiating measurement relying on ultrasound measurement. This is particularly advantageously for the patient, who is thus not exposed to harmful doses of radiation.

According to one embodiment of the invention, said two ultrasound images are a first image of an upper right-hand or left-hand zone of an individual's iliac bone and a second image of a lower zone of said iliac bone, said points of interest comprise an anterior-superior iliac spine and a pubic symphysis of said individual, and said device comprises means to determine a pelvic tilt of said individual on the basis of said relative spatial position of said points of interest.

Indeed, the measuring device of the invention can be applied particularly advantageously in the context of the measurement of a patient's pelvic tilt, i.e. the tilt of the pelvis relative to the vertical, this measurement being done in different positions of the patient (standing, seated, supine). To measure this tilt, it is enough to locate three known points of the pelvis (namely the two anterior-superior iliac spines and the pubic symphysis) defining the APP (anterior pelvic plane) which constitutes the reference plane relative to the patient to measure the pelvic tilt.

According to one aspect of the invention, said means for localizing said points of interest comprise means for processing said ultrasound images by segmentation capable of detecting said points of interest in said images. Such processing means therefore enable an automatic localizing of the anatomical landmarks without the practitioner's being required to take manual action. This advantageously reduces the time of use of the measuring device of the invention.

Such image-processing means comprise a set of processing operations common to both images, comprising especially operations of filtering, thresholding, conversion of intensity, etc.

They also comprise processing operations specific to each of the anatomical sites, given their particular geometrical features.

Thus, according to a first particular aspect of the invention, said means for processing said first image comprise means for identifying a longer segment in said first image, means for adjusting a parabola on said segment and means for detecting said point of interest as a vertex of said parabola. Such a processing enables an automatic detection of the anterior-superior iliac spine in the first image.

According to a second particular aspect of the invention, said means for processing said second image comprise means for identifying a segment in said second image, means for determining an axis of symmetry in said second image, means for adjusting a straight line on said segment and means for detecting said point of interest as an intersection of said axis of symmetry and of said straight line. The axis of symmetry is for example determined by using a method based on the Hough transform. Such a processing operation enables an automatic detection of the pubic symphysis on the second image.

According to one embodiment of the invention, such a measuring device comprises means of validation, by a user of said device, of said points of interest detected by said localizing means. Thus the practitioner can verify that the automatic localizing of the points of interest by the validation device is accurate, and validate it.

If this automatic localizing has failed, the practitioner can make a manual selection of the symphysis and/or of the iliac spine. Indeed, according to one embodiment of the invention, said localizing means comprise means for the selection of said points of interest on the screen by a user of said device. This manual selection can also be used by default, in one alternative embodiment, in place of the automatic detection of the anatomical landmarks.

According to one embodiment of the invention, said device comprises a screen enabling said ultrasound images to be viewed. Such a screen, which can be used to view the images of the anatomical sites acquired by the probes, also serves as an interface between the measuring device and the practitioner.

In one embodiment of the invention, such a screen is fixed to said support by an adjusting ball joint. Indeed, it should be possible to manipulate the measuring device in all positions of the patient: it is therefore important for the screen to be speedily pivotable so that the practitioner can keep it in his field of vision.

As a variant, the screen can consist of a tablet that is detachable from the support.

In one embodiment of the invention, at least one of said probes is connected to said support by a spherical link formed by a sphere that is fixedly attached to said probe, with a surrounding hollow structure that is also spherical, matching the shape of the probe and forming part of the frame. The orientation of the probe is deduced from information coming from an inertial measurement unit fixedly attached to this probe.

### 4. LIST OF FIGURES

Other goals, features and advantages of the invention shall appear more clearly from the following description of a preferred embodiment given by way of a simple illustratory and non-exhaustive example, made with reference to the appended drawings, of which:

FIG. 1 is an overall view of the portable ultrasound measuring device in one embodiment of the invention;

FIG. 2 is a schematic illustration of the kinematics of the measuring apparatus of FIG. 1;

FIGS. 3A to 3C show the position of the pelvic plane relative to a reference plane, respectively in a standing position (FIG. 3A), a supine position (FIG. 3B) and seated position (FIG. 3C);

FIG. 4 illustrates the three anatomical landmarks necessary to determine the anterior pelvic plane of FIGS. 3A to 3C;

FIGS. 5A and 5B illustrate the ultrasound capturing of an anterior-superior iliac spine (FIG. 5A) and a pubic symphysis (FIG. 5B) by means of the measuring apparatus of FIG. 1;

FIG. 6 presents a geometrical diagram of the device for measuring the pelvic tilt in one embodiment of the invention;

FIG. 7 presents a flow chart, in the form of a block diagram, of the implementing of the measuring device of FIG. 1;

FIG. 8 presents an example of a positioning of the inertial measurement units on the measuring apparatus of FIG. 1;

FIG. 9 illustrates an example of a positioning of a travel sensor on the support of the measuring apparatus of FIG. 1;

FIGS. 10A and 1013 present the details of the attachment of the ultrasound probes to the support of the measuring apparatus of FIG. 1;

FIG. 11 presents the portable measuring apparatus of FIG. 1 in its carrying case;

FIG. 12 is a view, in the form of a block diagram, of the electronic architecture of the measuring apparatus of FIG. 1.

### 5. DETAILED DESCRIPTION OF EMBODIMENTS OF THE INVENTION

The general principle of the invention relies on the designing of a portable ultrasound measuring apparatus comprising two ultrasound probes and an integrated system for measuring the position of the probes. The localizing of the anatomical points of interest in the ultrasound images, combined with knowledge of the position of the probes, makes it possible to determine the relative spatial position of the anatomical points of interest. When such a measuring apparatus is used to measure an individual's pelvic tilt, it therefore makes it possible to obtain a direct, precise and reproducible measurement of the pelvic tilt by using a single, non-irradiating, compact apparatus that can be easily and speedily used by the clinician.

Here below in this document, we shall strive to describe an embodiment of the invention in the context of the operation of total hip replacement surgery. The portable ultrasound measuring device of the invention can however be advantageously used for other medical applications.

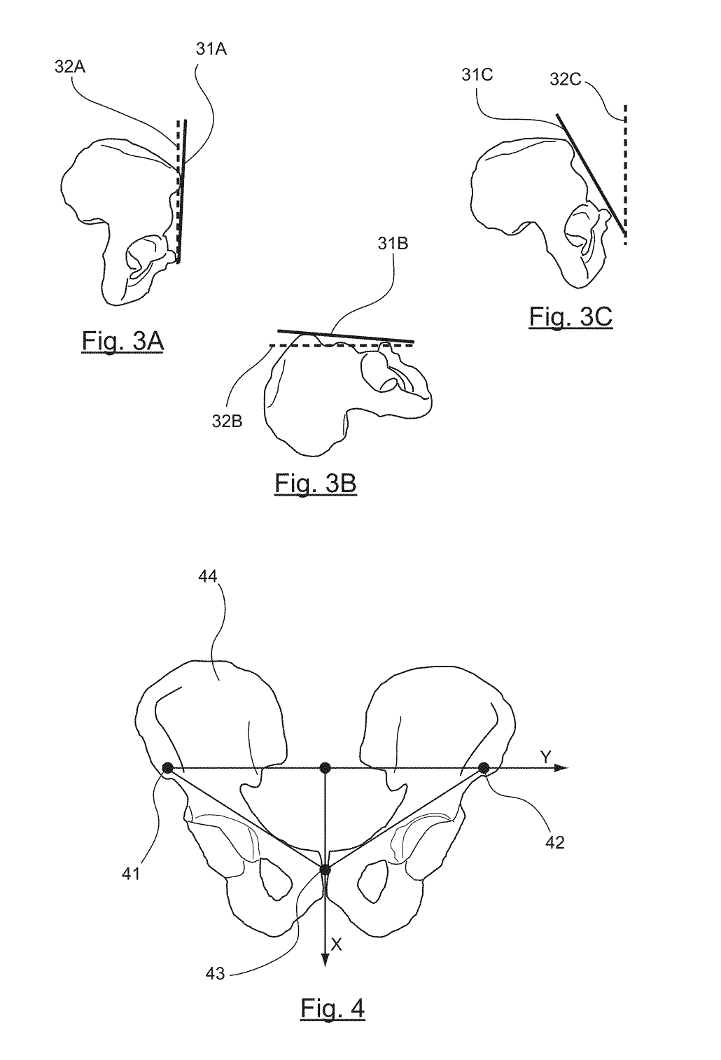

To begin with, FIGS. 3A to 3C and 4 serve to present a reminder of the definition of an individual's pelvic tilt. FIG. 4 presents the pelvic plane (XY) defined by the points corresponding to the two anterior-superior iliac spines 41 and 42 and to the pubic symphysis 43 on the iliac bone 44.

As can be seen in FIGS. 3A, 3B and 3C, the pelvic plane 31A, 31B, 31C can vary relative to a vertical or horizontal reference plane 32A, 32B and 32C. This dynamic behavior of the pelvis introduces modifications related to the functional orientation of the hip prosthesis and more particularly that of the acetabulum. It is therefore important to measure the pelvic tilt, i.e. the inclination of the patient's pelvic plane relative to the reference plane in different positions.

To this end, it is enough to locate three known points of the pelvis (namely the two iliac spines 41 and 42 and the pubic symphysis 43) defining the APP or anterior pelvic plan (XY).

Referring now to FIG. 1, an overall view is presented of the portable, ultrasound measurement device of the invention.

Such a measuring apparatus enables the easy and speedy measurement of the pelvic tilt in different positions of daily life, in order to integrate it into the scheduling of a total hip replacement operation. The use of such an apparatus must make it possible to reduce the number of operations of revision surgery and thus improve the quality of life of patients.

Such a measurement is done by means of the ultrasound probes during pre-operative consultation in at least three positions (for example the standing, seated and supine positions). When it is done by means of the portable ultrasound device in one embodiment of the invention, its main characteristics are that it is:
Reliable
Autonomous
Fast
Precise
Simple to use.

As illustrated in FIG. 1, the ultrasound apparatus consists of a support 10, which takes the form of an arm, and two ultrasound probes 11₁ and 11₂ mounted on the support 10. A screen 12 is also integrated into the ultrasound apparatus for the purpose of viewing the images of the anatomical sites acquired by the probes. This screen also serves as an interface between the apparatus and the practitioner. For this screen 12 to be speedily pivotable, and so that the user can have it permanently in his field of vision, it is mounted on the support 10 by means of an adjusting ball joint, comparable for example to that of a camera tripod.

In addition, the probes 11₁ and 11₂ are movable relative to each other along a slide link, to enable the practitioner to adjust the distance between them. Moreover, they are mounted relative to the support 10 with a ball-joint link for the probe 11₁, and with a ball-joint link and slide link for the probe 11₂.

It is indeed necessary that the two probes should be easily adaptable to the patient's morphology in all three positions, standing, seated and supine, whatever the patient's body mass. Preferably, the spacing between the probes 11₁ and 11₂ is chosen so that it can vary between about 10 cm and 25 cm.

The apparatus is handled by taking the probes 11₁ and 11₂ directly by hand. Thus, the mechanism of the apparatus (support 10, screen 12 and hinges) are situated above the practitioner's hands and therefore do not hamper the handling of the apparatus.

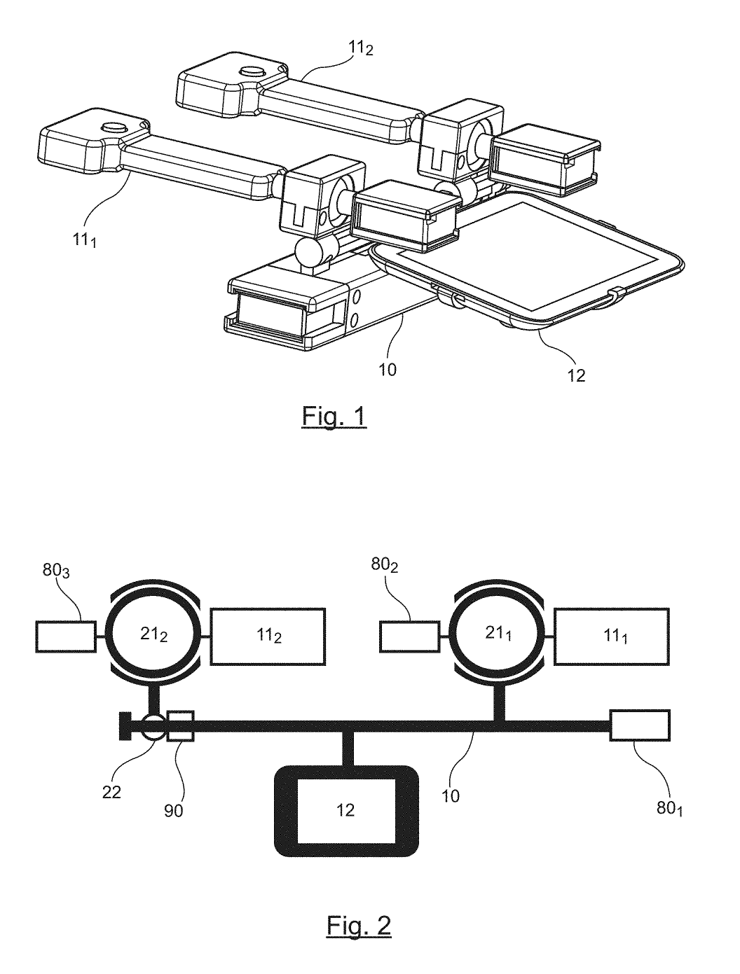

FIG. 2 gives a schematic view of the kinematics of the apparatus of FIG. 1. The kinematic capacities of the ball-joint socket links between the probes 11₁ and 11₂ and the support 10 are created by means of links 21₁ and 21₂ with concave and convex spherical surfaces. It is indeed desirable that the probes should have six degrees of freedom relative to each other.

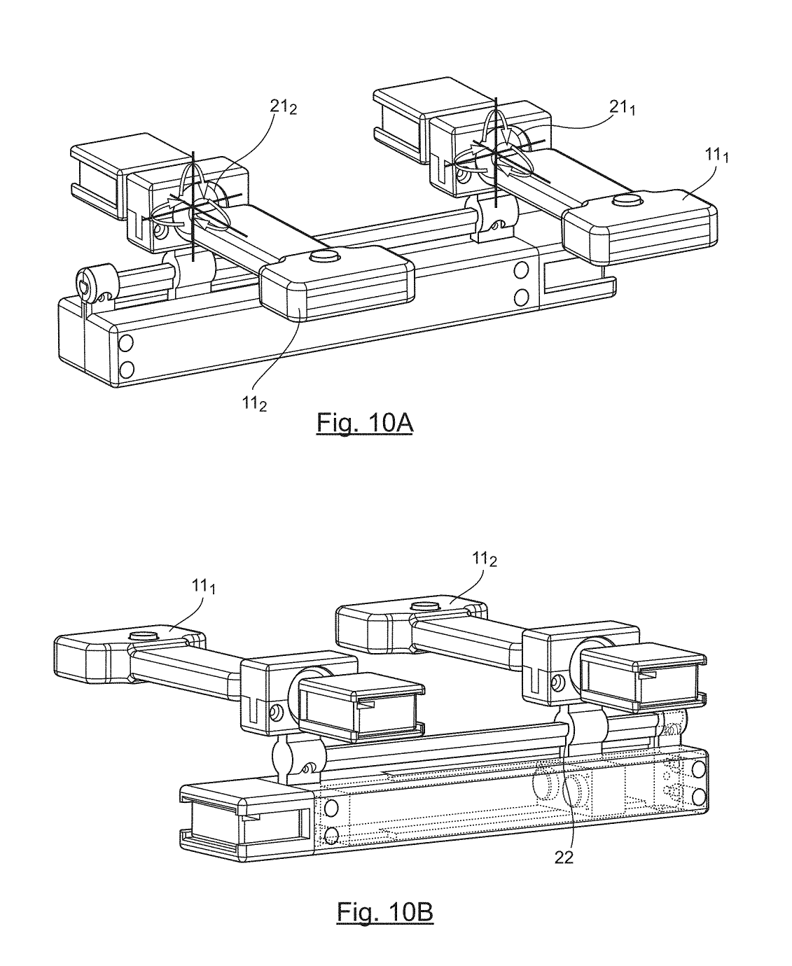

FIGS. 10A and 1013 provide a more detailed illustration of an embodiment of this kinematics. Thus, the mobility of the probe 11₁ relative to the support 10 is ensured by means of the ball-joint link 21₁ while the mobility of the probe 11₂ relative to the support 10 is ensured by means of the ball-joint link 21₂ and a slide link 22. This slide link referenced 22 ensures the translation between the right-hand and left-hand parts of the apparatus.

This architecture enables the practitioner to easily adjust the probes to the anatomical sites of interest for the measurement of pelvic tilt, namely the pubic symphysis and the iliac spines. This architecture is moreover compact, robust and stable.

In addition, in order to localize the two probes 11₁ and 11₂ relative to each other, the orientation and the distance between the two probes must be measured. It is indeed necessary to know the position of the two probes relative to each other when the practitioner is capturing the ultrasound images.

The embodiment of FIG. 1 thus provides for three orientation sensors, also called inertial measurement units, fixedly attached to the probes 11₁ and 11₂ and to the support 10. Such sensors are, for example, inertial measurement units by OMNI Instruments (registered mark) of the LPMS-B motion sensor type. These instruments are compact and robust.

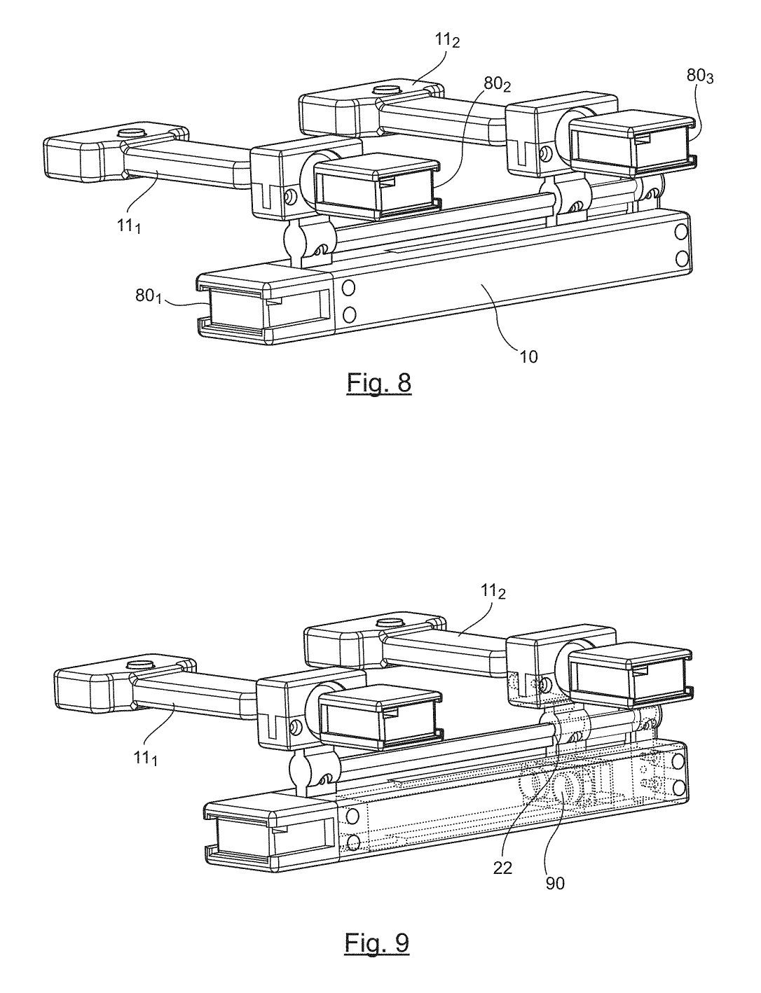

One solution for the position of the inertial measurement units 80₂ and 80₃ fixedly attached to the ultrasound probes 11₁ and 11₂ is illustrated in FIG. 8.

The translation between the two probes 11₁ and 11₂ is measured by means of a travel sensor 90, illustrated in FIG. 9. Such a travel sensor is for example the HC-SR04 (registered mark) ultrasonic sensor module which comprises an ultrasonic transmitter and receiver and deduces distance from the time of travel of the ultrasound. In the embodiment of FIG. 9, the transmitter and the receiver are fixedly attached to the element supporting the ball element of one of the probes. This element slides (slide link 22) in a chamber. The wall of this chamber, opposite that of the sensor reflects the ultrasounds. When the operator adapts the apparatus to the patient, he applies forces to each part of the apparatus, thus causing a translation of the two parts of the ultrasound measuring apparatus relative to each other, and enabling the sensor to detect the travel.

Finally, in order to know the position of the ultrasound measurement apparatus of the invention relative to the vertical, this apparatus also comprises an inertial measurement unit 80₁, illustrated in FIG. 8. Such an inertial measurement unit is for example of the LPMS-B (registered mark) motion sensor type by OMNI Instruments. This instrument has very high 3D precision and is very compact. Such an inertial measurement unit 80₁ can be placed at any point whatsoever of the structure of the apparatus 10. FIG. 8 illustrates an example of positioning of this inertial measurement unit 80₁, which does not get in the way during handling and provides load-balancing for the apparatus.

The portable ultrasound apparatus of FIG. 1 must furthermore comprise an information-processing system that integrates the data coming from the position and orientation sensors described here above, and the position of the anatomical sites located in the ultrasound images, as described in greater detail here below. Such a processing system comprises especially one or more analyzers cooperating with the ultrasound probes 11₁ and 11₂ and an electronic calculator or computer.

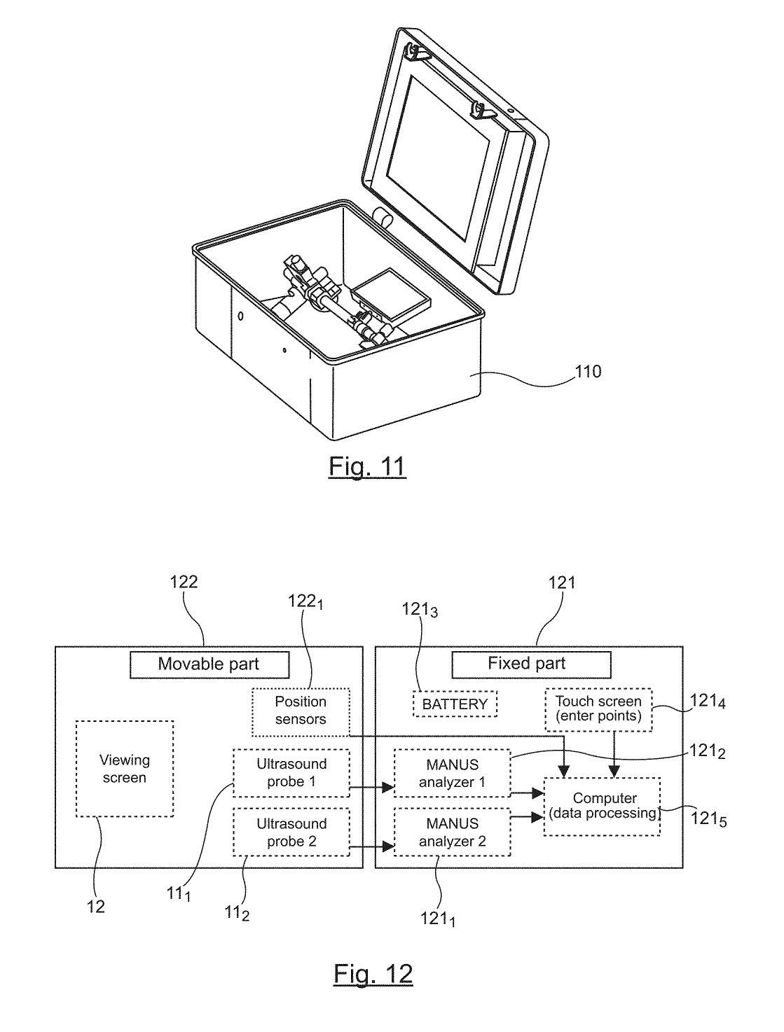

The assembly is easy to transport for use in medical consultation, as illustrated in FIGS. 11 and 12. A case 110 serves on the one hand as a fixed stand to be placed beside the patient and on the other hand as a carrying case. It contains the fixed part 121 of the ultrasound measuring apparatus, namely the analyzers 121₁ and 121₂, as well as a battery 121₃ (or electrical transformer), the electronic computer 121₃, and a screen 121₄. It is connected to the movable part 122 illustrated in FIG. 1 by a cord. A large touch screen 121₄ fixed to the lid of the case 110 is used to enter the anatomical points with high precision as described in greater detail here below. The movable part 122 of FIG. 1 is light (weighing about one kilogram or less), making its handling easy and precise. As already described with reference to FIG. 1, this mobile part comprises the viewing screen 12, the ultrasound probes 11₁ and 11₂ and the position sensors 122₁ (namely the inertial measurement units 80₁, 80₂ and 80₃ as well as the travel sensor 90).

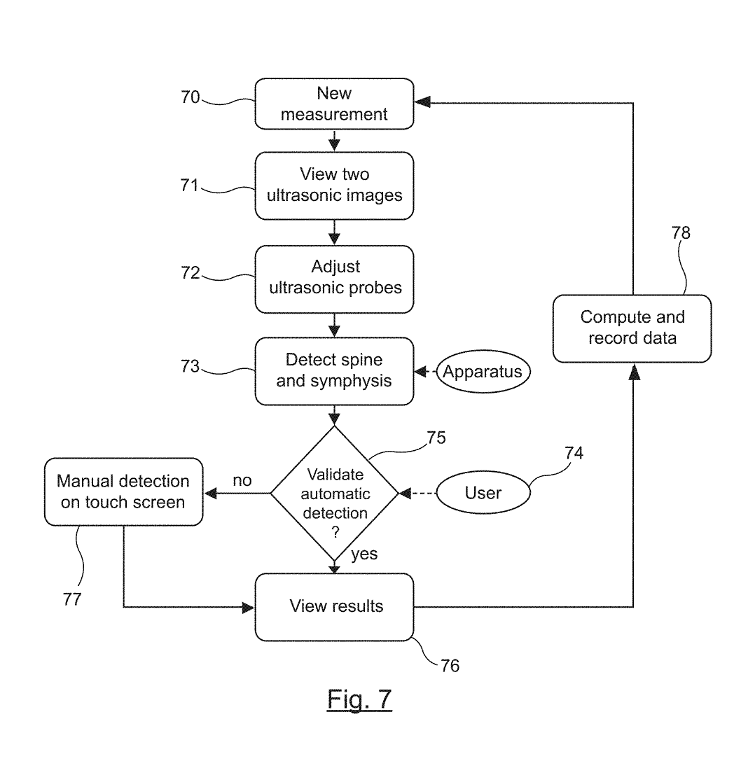

Referring now to FIG. 7 we describe a flowchart of operation of the ultrasound measurement apparatus described here above.

During a medical consultation preparatory to a total hip replacement operation, the practitioner applies the ultrasound probes 11₁, 11₂ to the patient in order to simultaneously locate the pubic symphysis 43 and one of the anterior-superior iliac spines 41 or 42. Once these anatomical sites have been located, the practitioner launches the processing sequence which will integrate all the information coming from the different sensors 122₁ integrated with the ultrasound measurement device enabling the computation of the pelvic tilt.

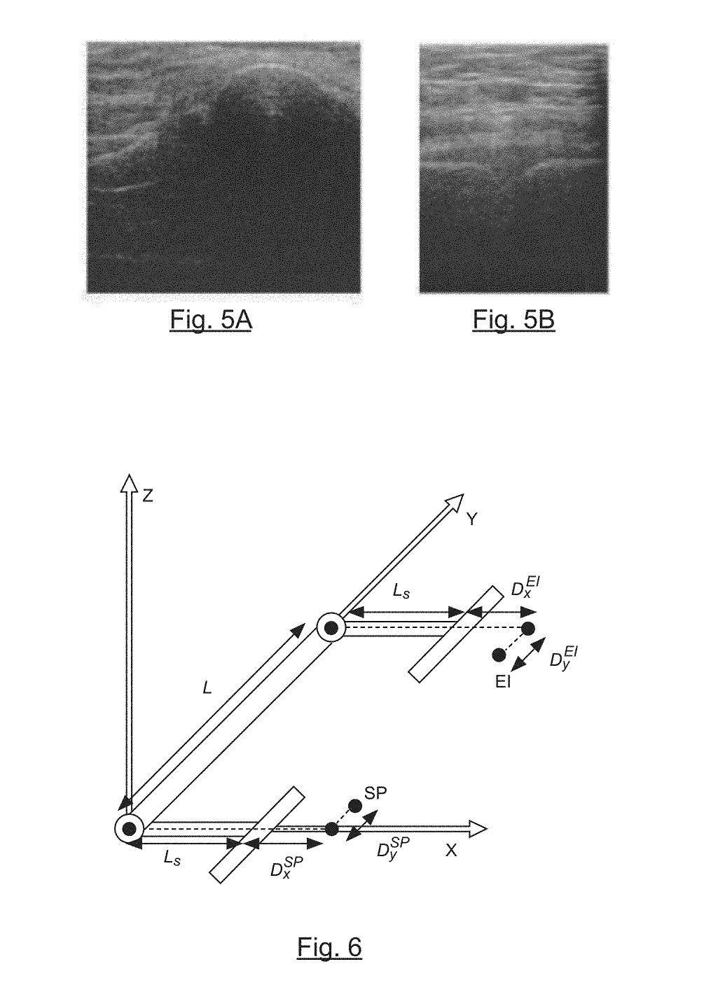

Thus, when a new measurement 70 is started, the practitioner first of all adjusts the ultrasound probes 11₁, 11₂ mounted on ball-joint links and mutually hinged by means of a slide link, in order to place them so that they face the anatomical sites of interest 41, 42, 43. He then views 71 the images obtained by means of the control screen 12, and adjusts 72 the position of the probes more finely if necessary. He validates these acquisitions when they enable him to distinguish the pubic symphysis 43 (FIG. 5B) and an anterior-superior iliac spine 41 or 42 (FIG. 5A).

The following step referenced 73 is that of the automatic treatment of the image, which makes it possible to achieve the automatic location of the anatomical sites of interest constituted by the pubic symphysis 43 and the iliac spines 41, 42.

A common processing base is first of all applied to the two images (of the pubic symphysis 43 and of one of the spines 41, 42); it is followed by processing operation specific to each of the anatomical sites taking account of their special geometrical features.

The basic processing of the ultrasound images can, for example, be broken down as follows:
Anisotropic filtering
Otsu thresholding
Transformation of intensity
<<South Shadow>> Filtering
Canny filtering
Preservation of the last segmented line on each column of the image
Operations of mathematical morphology

The specific final processing operations are the following:
for the iliac spine 41, 42 (FIG. 5A), the longest segment is kept and a parabola is adjusted to it. Its vertex will localize the anatomical point of interest.
for the pubic symphysis 43 (FIG. 5B), an axis of symmetry is determined by using, for example, a method based on the Hough transform, a straight line is then adjusted to the previously obtained contour of the symphysis. The position of the anatomical point of interest consists of the intersection of this straight line with the axis of symmetry.

In the course of a step referenced 75, the user 74 validates or does not validate the automatic detection of the anatomical reference markers of interest operated by the apparatus during the step referenced 73.

If this automatic detection is validated, the user 74 views the results on the screen, during a step referenced 76.

If not, the user 74 can make a manual selection 77 of the symphysis 43 and/or of the iliac spine 41, 42 on the touch screen 121₄.

The system of the invention then records the set of data and computes the pelvic tilt during a step referenced 78.

To this end, when the anatomical sites of interest have been located in the ultrasound images acquired by the probes, the system determines their relative spatial positions from the data delivered by the inertial measurement units, fixedly attached to the probes and the support, and by the translation sensor integrated into the slide link.

The geometrical principle of the computation of the pelvic tilt is illustrated in FIG. 6. The pelvic tilt could be computed for example by using the following formula:

$$Pelvic tilt = acos ( \frac{{( \vec{{v}_{SP - EI}} )}_{z}}{\sqrt{{( \vec{{v}_{SP - EI}} )}_{x}^{2} + {( \vec{{v}_{SP - EI}} )}_{y}^{2} + {( \vec{{v}_{SP - EI}} )}_{z}^{2}}} )$$

With: \vec{v_{SP-EI}}=[0 L 0]·R_{M}+[L_{S}+Dₓ^{EI} D_{y}^{EI} 0]·R_{S}^{EI}−[L_{S}+Dₓ^{SP} D_{y}^{SP} 0]·R_{S}^{SP},
where the different variables, measured at the time of the validation of the ultrasound images by the user are:
L: the distance between the two ultrasound probes 11₁ and 11₂, measured by the distance sensor 90.
L_{S}: the length of the ultrasound probe (distance between the center of the ball-joint link and the extremity of the probe 11₁, 11₂).
R_{M}: a matrix containing the roll, pitch and yaw motions given by the inertial measurement unit 80₁ mounted on the structure 10.
R_{S}^{EI}: a matrix containing the roll, pitch and yaw motions given by the inertial measurement unit 80₃ mounted on the probe 11₂ locating the iliac spine EI 41 or EI 42.
R_{S}^{SP}: a matrix containing the roll, pitch and yaw motions given by the inertial measurement unit 80₂ mounted on the probe 11₁ locating the pubic symphysis SP 43.
Dₓ^{EI}: the abscissa value of the point representing the iliac spine EI 41 or EI 42, detected on the image by segmentation.
D_{y}^{EI}: the ordinate value of the point representing the iliac spine EI 41 or EI 42, detected on the image by segmentation.
Dₓ^{SP}: the abscissa value of the point representing the pubic symphysis SP 43, detected on the image by segmentation.
D_{y}^{SP}: the ordinate value of the point representing the pubic symphysis SP 43, detected on the image by segmentation.
\vec{v_{SP-EI}}: the vector connecting the iliac spine EI 41 or EI 42 with the pubic symphysis SP 43 in space.

The portable ultrasound measuring apparatus resolves the problems of low precision or those linked to the invasive methods of measurement of the pelvic tilt as well as the autonomy, portability and ease of use of the equipment needed for this measurement. It enables especially:
the acquisition and the viewing of the two simultaneous ultrasound images (the term <<simultaneous>> is herein understood to mean two images acquired at the same instant or at instants close enough to each other for the patient not to have moved between the acquisitions of the two shots);
the automatic segmentation of the ultrasound images and the automatic detection of the anatomical sites of interest;
the spatial locating of areas of interest through a system of measurement of positioning of the probes;
the integrated computation of the pelvic tilt.

The method of measurement proposed by the present invention is non-irradiating and the precision of the measurement, estimated by simulation, shows a mean standard deviation of about 1.9°, which is comparable to that obtained by Dardenne et al.

1. An ultrasound measuring device comprising:
a support bearing first and second ultrasound probes movable relative to each other by slide link, each of the first and second probes being movable relative to the support by ball-joint link, said probes being capable of simultaneously acquiring first and second ultrasound images;
a first set of measuring elements for measuring a relative positioning of said probes, comprising a travel sensor and at least two orientation sensors;
a second set of measuring elements for measuring a positioning of said device relative to a reference plane, comprising at least one orientation sensor;
a localizer, which is configured to localize at least one point of interest on each of said first and second ultrasound images; and
a processing device, which is configured to process data coming from said first and second sets of measuring elements and deliver a relative spatial position of said at least one point of interest located in said first and second ultrasound images.

2. The ultrasound device according to claim 1, wherein said first and second ultrasound images comprise a first ultrasound image of an upper right-hand or left-hand zone of an individual's iliac bone and a second ultrasound image of a lower zone of said iliac bone, respectively,
and said at least one point of interest comprises an anterior-superior iliac spine and a public symphysis of said individual,
and said device comprises means to determine a pelvic tilt of said individual on the basis of said relative spatial position of said at least one point of interest.

3. The ultrasound device according to claim 1, wherein said localizer for localizing said at least one point of interest comprises means for processing said ultrasound images by segmentation capable of detecting said at least one point of interest in said ultrasound images.

4. The ultrasound device according to claim 3, wherein said means for processing said first ultrasound image comprise means for identifying a longer segment in said first ultrasound image, means for adjusting a parabola on said segment and means for detecting said at least one point of interest as a vertex of said parabola.

5. The ultrasound device according to claim 3, wherein said means for processing said second ultrasound image comprise means for identifying a segment in said second ultrasound image, means for determining an axis of symmetry in said second ultrasound image, means for adjusting a straight line on said segment and means for detecting said point of interest as an intersection of said axis of symmetry and of said straight line.

6. The ultrasound device according to claim 3, further comprising comprises means of validation, by a user of said device, of said at least one point of interest detected by said localizer.

7. The ultrasound device according to claim 1, wherein said ultrasound device comprises a screen enabling said ultrasound images to be viewed.

8. The ultrasound device according to claim 7, wherein said screen is fixed to said support by an adjusting ball joint.

9. The ultrasound device according to claim 1, wherein the localizer comprises means for selecting said at least one point of interest on the screen by a user of said device.

10. The ultrasound device according to claim 1, wherein at least one of said probes is connected to said support by a spherical link.

11. The ultrasonic device of claim 1, wherein:
the support bears two ultrasonic probes, comprising the first and second ultrasound probes; and
the first set of measuring elements comprises one travel sensor, recited in claim 1.
